(19) United States
(12) Patent Application Publication
(10) Pub. No.: US 2012/0252104 A1
(43) Pub. Date: Oct. 4, 2012

(54) ENERGY EFFICIENT TEMPERATURE CONTROL OF ENCLOSED MICROALGAE CULTIVATOR

(75) Inventors: Brian J. Waibel, Kennett Square, PA (US); Sam Couture, Philadelphia, PA (US)

(73) Assignee: Elemen Cleantech, Inc., Newark, DE (US)

(21) Appl. No.: 13/502,924

(22) PCT Filed: Oct. 25, 2010
(86) PCT No.: PCT/US2010/053980

§ 371 Date: Jun. 14, 2012

Related U.S. Application Data
(60) Provisional application No. 61/254,841, filed on Oct. 26, 2009.

(51) Int. Cl. C12N 1/12; C12M 1/04

(52) U.S. Cl. 435/257.1; 435/303.1

(57) ABSTRACT

The present invention provides methods and systems for energetically efficient and economically viable temperature regulation of the environment within commercial scale enclosed microalgae cultivators.

### CROSS-REFERENCE TO RELATED APPLICATIONS

This application is a U.S. national phase filing under 35 U.S.C. §371 of International Appl. No. PCT/US2010/053980, filed on Oct. 25, 2010, which claims the benefit of U.S. Provisional Application No. 61/254,841, filed on Oct. 26, 2009, the entire contents of which are hereby incorporated herein by reference in its entirety for all purposes.

### FIELD OF THE INVENTION

The present invention provides energy efficient methods and systems for temperature regulation of a microalgae culture in an enclosed cultivator.

### BACKGROUND OF THE INVENTION

The phototrophic production of algae uses light input, carbon dioxide, and nutrients to create biomass comprised of proteins, carbohydrates, and lipids. Per Pulz and Schiebenbogen, there are two broad types systems to cultivate algae: open cultivation systems and closed and semi-closed photobioreactors (“PBRs”). See, Pulz and Schiebenbogen, “Photobioreactors: Design and Performance with Respect to Energy Input,” in Advances in Biochemical Engineering/Biotechnology, T. Scheper, ed., (1998) 59:123-151, Springer-Verlag. Open cultivation systems include natural or artificial ponds, raceways, and inclined surface systems. Ponds, which receive contamination of various kinds and invasion of other species are limited to growing alga species that are a combination of fast growing, naturally occurring, or extremophiles. An extremophile is an organism that is particularly adapted to a unique environment, such as one having either extreme low or high temperatures or abnormally low or high pH. Ponds have thermal regulation limited to natural evaporation and, further, lack a stirring mechanism to facilitate introduction of CO₂.

Raceway systems are oblong shaped cultivators typically divided into two parallel lanes with a fluid propulsion means at one or more locations and a turn around at the opposite end. The raceway is typically a completely open system, such as those employed by Earthrise Farms for Spirulina production or in Israel for the production of Dunaliella (See, Sheenan, et al., “A Look Back at the U.S. Department of Energy's Aquatic Species Program: Biodiesel from Algae” National Renewable Energy Laboratory, US Department of Energy, NREL/TP-580-24190, July 1998). These systems include features for fluid propulsion, for the introduction and mixing of CO₂, for the introduction of makeup water and nutrient solution, for the removal of culture for harvesting the algae. The most typical propulsion means is a paddlewheel; although, E. A. Laws (1983) at the University of Hawaii has employed an air lift. See, Laws, et al., “A Simple Algal Production System Designed to Utilize the Flashing Light Effect,” Biotechnology and Bioengineering (1983) 25:2319-2335.

Raceway systems are made at a variety of different scales. A typical test cultivator would be between 15 and 150 m². Scale-up cultivators would be in the range of 150 to 400 m². A commercial production raceway cultivator would typically be in the range of 400 to 3000 m². Culture depth in a raceway is generally between 15 and 30 cm. Flow velocities within the cultivator are in the range from 10 to 40 cm/sec, with a value of 25 cm/sec being typical. Thus, a commercial scale raceway would be filled with about 60,000 L to 900,000 L of algae culture and, more typically, would be between about 75,000 L and 400,000 L. The surface to volume ratio for a commercial structure varies from about 3 to 10 m⁻¹. With a higher surface to volume ratio, growth rate is improved. A lower surface to volume ratio reflects a deeper culture depth and is indicative of higher light shadowing through the fluid column. A typical volumetric productivity in a raceway growing Chlorella would be about 0.1 to 0.2 g/(L day). A culture density at harvest would be in the range of about 1.0 to 2.0 g/L.

Richmond (1992) put the challenge associated with raceways very succinctly:
“This mode of production suffers usually from many weaknesses, since it does not permit a satisfactory response to the two major variables that limit productivity outdoors—i.e. —solar irradiance and ambient temperature . . . . The drawbacks of the open system related in essence to the lack of temperature control and the long light-path which dictates maintenance of disadvantageously low cell concentrations.”

See, abstract of Richmond, et al., “Open systems for the mass production of photoautotrophic microalgae outdoors: physiological principles,” J Appl Phycol (1992) 4:281-286. In the same publication, Richmond indicates that there is a clear interaction between solar irradiance and open raceway temperature. Richmond states that “Maximum utilization of solar energy can be achieved only when the temperature is optimal. However, the usual case in outdoor open systems is that temperature is not optimal, and thus the full potential imbued in the PFD (supersaturating photon densities) for the photosynthetic reactions cannot be expressed.” See, Richmond, J Appl Phycol at page 283. Furthermore, it is noted that open raceways are poor in responding to both annual and diurnal fluctuations in temperatures.

The conventional wisdom in phycology is that raceways are, almost by definition, open. Hase (2000) documents the use of a raceway cultivator within a greenhouse to limit temperature fluctuations due to low ambient temperatures (greenhouse is for heat retention), avoid microbial contamination in rainfall, and improve CO₂ utilization. See, Hase, et al., “Photosynthetic Production of Microalgal Biomass in a Raceway System under Greenhouse Conditions in Sendai City” J Biosci Bioeng, (2000) 89:157-163. The cultivator in this study, at 0.986 m², is laboratory size. In the June to August timeframe, the papers notes the average daily maximum temperature of ambient (outside) to be 24.2° C., the greenhouse (interior) to be 35.9° C., and a culture of Chlorella sp. an average daily maximum temperature of 26.5° C. It is noted that this attempt to enclose a raceway within a greenhouse made no attempt to actively control the raceway's culture temperature. Despite the small scale of the demonstration system, the paper captures the value of a greenhouse enclosure—“Although the construction of a greenhouse is an additional cost factor, microalgal production would become stable and efficient under greenhouse conditions.”

Becker, in his chapter on large-scale cultivation pond design, describes a cultivator in China “roofed by iron frames covered by transparent plastic sheets.” The interior of the cultivator is shown in FIG. 10.21. The intent of the covering was to extend the cultivation season in areas with temperate climate conditions. No attempt was made to regulate the temperature by modulating the rate of evaporation. See, Becker, et al., in Microalgae: Biotechnology and Microbiology, Cambridge Studies in Biotechnology, Ed.: Baddiley, Carey, Higgins, and Potter, Cambridge University Press, Cambridge (1994).

Growth rates in open raceways are dependent on species selection, light conditions, and ambient temperature. To some extent, natural evaporation counteracts solar heating. In conventional raceway phycology, thermal regulation is limited to natural evaporation. The field's approach has been to leverage extremophile species that can tolerate the ambient temperature fluctuations as minimally tempered by natural evaporation. Productivity rate of 15-25 g/(day m²) are noted in the tropics and California, and 12-15 g/(day m²) in Central Europe and Asia. With agitation due to an immersed foil and appropriate harvest and dilution of the culture, Laws documented productivity rates with Tetraselmis suecica of greater than 50 g/(day m²). See, Laws E A, Taguchi S, Hirata J, Pang L, “High Algal Production Rates Achieved in a Shallow Outdoor Flume, Biotech Bioeng (1986) 28:191-197.

Inclined surface system was originally developed by Setlik (1970) in the Czech Republic. See, Setlik, et al., Alol Stud (Trebon) (1970) 1:111. These systems involve a series of level terraces of a defined inclination. The culture thickness is intentionally low, on the order of 1 cm, and intentionally turbulent to stir the culture and prevent shadowing. These systems have a higher surface to volume ratio, e.g., on the order between 20 and 100 m⁻¹. Because the amount of surface relative to the culture volume, these systems are capable of growing cultures at higher densities (on the order of about 10 g/L) and higher volumetric productivities (0.95 g/(L day)) than raceways. A variant on this cultivator design is further documented in U.S. Pat. No. 5,981,271 to Doucha, et al. They describe the ability to grow algae to culture concentrations of 20 to 30 g/L, a culture density more common in the heterotrophic cultivation in fermenters. Furthermore, Grobbelaar, et al., in “Variation in some photosynthetic characteristics of microalgae cultured in outdoor thin-layered sloping reactors,” J App Phycol (1995) 7:175-184, examines the tradeoffs between the use of thin-layered smooth sloping cultures (TLSS) having a culture depth of 5-7 mm and thin-layered baffled sloping cultures (TLBS) having a culture depth of 5-15 mm. In this study with Scenedesmus obliquus and Chlorella spp., culture densities in excess of 10 g/L were readily attained with both TLSS and TLBS systems.

Closed and semi-closed photobioreactors (PBR) come in a large variety of configurations. Some of the more common configurations are clear tubular systems, parallel glass plate PBRs, and plastic film cultivators. The tubular systems have been used by Pulz at IGV in Germany and Alga Technologies in Israel. The use of parallel glass plate PBRs has demonstrated by Trota, Tredici and Materassi, Pulz, Richmond, and Grobbelaar. See, Trota, Aquaculture, (1981) 22:283; Tredici, et al., “Fully-Controllable Photobioreactors,” ECB6: Proceedings of the 6th European Congress on Biotechnology, Florence (1994) p 1011; Pulz, “Cultivation Techniques for Microalgae in Open and Closed Ponds”. Proceedings of the 1st European Workshop on Microalgal Biotechnology, Potsdam-Rehbruecke, Germany (1992) p 61; Richmond, et al., “Optimization of a flat plate glass reactor for mass production of Nannochloropsis sp. Outdoors,” J Biotech, (2001) 85:259-269; and Grobbelaar, et al., “Use of photoacclimation in the design of a novel photobioreactor to achieve high yields in algal mass cultivation” J Appl Phycol, (2003) 15:121-126. Plastic film cultivators are typified by the G3 PBR design of Solix Biofuels. See, Lehr and Posten, “Closed photo-bioreactors as tools for biofuel production” Current Opinion in Biotechnology, (2009) 20:1-6.

Per Pulz (1992) supra, closed PBRs provide a number of advantages:

Efficient CO₂ usage

Mitigate contamination risk

Thermal regulation

Controlled hydrodynamics

Repeatable cultivation conditions

Higher tolerance for environmental influences

Smaller space requirements

Closed PBRs typically provide higher productivity rates than open raceway systems. They also come at substantial capital cost due to the system elements required to retain the algae, provide appropriate turbulence, modulate light exposure, regulate temperature, introduce CO₂, and remove O₂. Operational costs also tend to be high due to the higher pumping energy.

U.S. Pat. No. 6,579,714 to Hirabayashi, et al., documents a spherical closed PBR. It shows an inner and outer clear hemispherical shell. Thermal control, as is a typical mechanism in closed PBRs, is achieved by spraying cooling water on the surface of the PBR. Evaporative cooling results in thermal regulation of the culture. While not specifically documented in this patent, it is noted that such a spray method with typical, mineral laden water results in the accumulation of dissolved solids on the PBR surface that lead to light limiting for the culture and a later requirement for cleaning

For over fifteen years since the 1992 Pulz review, the phycology field has recognized that raceway cultivators can be built at substantial scale, that they are open systems, are limited to cultivating a few extremophile species, and that the uncontrolled factors in raceways cannot be readily addressed. The field has attempted to develop an almost endless array of variations on closed PBR designs. The field, fundamentally, has not revisited the physics and facts that prevented the isolation of the raceway from the greater ambient environment, the thermal conditions within the culture, CO₂ gas uptake, and O₂ off gassing to prevent oxygen inhibition. The field has chased every strategy for higher productivity levels (i.e. g/(m² day)) of closed PBRs while failing to recognize the optimization function is not productivity in isolation but, rather, productivity per unit capital cost. Raceways offer lower capital cost per unit cultivation area than closed PBRs. Furthermore, the field has assumed that raceways are most suited for the cultivation of extremophiles such as Spirulina, Chlorella, and Dunaliella that grow in highly selective environments that are toxic to other algae and protozoa. Spirulina requires an alkaline environment, Chlorella requires a nutrient rich media, and Dunaliella requires very high salinity. Borowitzka (1999) states that species appropriate for aquaculture nutrition (e.g. Skeletonema, Chaetoceros, Thalassiosira, Tetraselmis, and Isochrysis) must be grown in a closed (i.e., a PBR) system. See, Borowitzka, “Commercial production of microalgae: ponds, tanks, tubes and fermenters”, J Biotech, (1999) 70:313-321.

Other than the Hase (2000) reference (supra) to a greenhouse enclosure of an experimental raceway and the Becker reference (supra), the phycology literature has few references enclosing a raceway at commercial scale (i.e., greater than 400 m²). The NREL Aquatic Species Program summary report (Sheehan 1998) and Laws (1983) document heating and cooling of an open raceway with a conductive heat exchanger, which would require prohibitive costs for temperature regulation at a commercial scale. See, Sheehan, et al., “A Look Back at the U.S. Department of Energy's Aquatic Species Program: Biodiesel from Algae” National Renewable Energy Laboratory, U.S. Department of Energy, NREL/TP-580-24190, July 1998; and Laws, Biotechnology and Bioengineering (1983) 25:2319-2335.

### BRIEF SUMMARY OF THE INVENTION

The present invention provides energy efficient methods and systems for regulating the temperature of a commercial scale, e.g., at least about 60,000 L, aquatic microalgae culture in an enclosed cultivator. The methods and systems utilize evaporative cooling by spraying the microalgae culture itself within the airspace of the enclosed cultivator. By regulating airflow speed in the airspace within the cultivator and above the aquatic microalgae culture and/or pump speed of the microalgae culture through the sprayer, the amount of cooling effected by evaporation can be regulated or controlled. The sensors, mixers, fans and pumps, and other mechanisms requiring energy input in the present enclosed microalgae cultivator systems can be powered by the output current of a photovoltaic cell or an array of photovoltaic cells. Generally, the methods and systems do not involve or utilize conductive heat transfer to regulate or control the temperature of the aquatic microalgae culture.

Accordingly, in one aspect, the invention provides methods of regulating the temperature of an aquatic microalgae culture in an enclosed cultivator. In some embodiments, the methods comprise the steps of:

a) maintaining airflow in the airspace within the enclosed cultivator above the aquatic microalgae culture by taking in air from a source external to the enclosed cultivator and exhausting air from the airspace above the aquatic culture to the outside the enclosed cultivator, thereby effecting evaporative cooling; and

b) spraying the aquatic microalgae culture in the airspace above the aquatic microalgae culture; thereby increasing the surface area of water exposed to the airflow and enhancing the effect of evaporative cooling, whereby the temperature of the aquatic microalgae culture in the enclosed cultivator is regulated.

In a related aspect, the invention provides methods of enhancing evaporative cooling of an aquatic microalgae culture in an enclosed cultivator. In some embodiments, the methods comprise spraying the aquatic microalgae culture in the airspace above the aquatic microalgae culture; thereby enhancing the effect of evaporative cooling.

With respect to the embodiments of the methods, in some embodiments, the aquatic microalgae culture is maintained at a temperature at or below about 35° C. In some embodiments, the aquatic microalgae culture is maintained at a temperature in the range of about 15° C. to about 35° C.

In some embodiments, the aquatic microalgae culture has a growth area of at least about 400 m², for example, at least about 500 m², 600 m², 700 m², 800 m², 900 m², 1000 m², 1200 m², 1500 m², 1800 m², 2000 m², 2500 m² or 3000 m², or more. In some embodiments, the aquatic microalgae culture has a growth area in the range of about 400 m² to about 3000 m².

In some embodiments, the aquatic microalgae culture has a volume of at least about 60,000 L, for example, at least about 75,000 L, 100,000 L, 150,000 L, 200,000 L, 250,000 L, 300,000 L, 350,000 L, 400,000 L, or more. In some embodiments, the aquatic microalgae culture has a volume in the range of about 60,000 L to about 900,000 L, for example, in the range of about 75,000 L to about 400,000 L.

In some embodiments, the enclosed cultivator is a raceway system. For example, in some embodiments, the enclosed cultivator has a width of at least about 16 meters and a length of at least about 100 meters. In some embodiments, the enclosed cultivator has a width of at least about 24 meters and a length of at least about 150 meters. In some embodiments, the enclosed cultivator has an length:width aspect ratio of about 6.25:1.

In some embodiments, the enclosed cultivator is a thin-layered sloping reactor.

In some embodiments, the aquatic microalgae culture is sprayed through a nozzle that expels a cone of liquid at a spray angle from about 30° to about 170°, for example, at an angle of about 30°, 60°, 90°, 120°, 150°, 170°, or 180°, as appropriate.

In some embodiments, the flow rate of the microalgae culture through the one or more pumps and spray nozzles is in the range of about 10 L/min to about 1,000 L/min, for example, about 10 L/min, 20 L/min, 50 L/min, 100 L/min, 200 L/min, 400 L/min, 600 L/min, 800 L/min or 1000 L/min.

In some embodiments, the methods further comprise the step of determining the ambient temperature and/or the relative humidity within the enclosed cultivator before spraying the aquatic microalgae culture.

In some embodiments, the flow of the air in the airspace above the aquatic microalgae culture and/or the flow of the spraying are powered by the output current of a photovoltaic cell.

In some embodiments, the microalgae in the culture are Selenestrum, Scenedesmus, Nannochloropsis or Isochrysis.

In a further aspect, the invention provides an enclosed cultivator system. In some embodiments, the enclosed cultivator system comprises:

a) a covering over a reservoir, wherein the reservoir is configured for maintaining an aquatic microalgae culture;

b) one or more inlets in the covering, wherein the inlets comprise a fan for taking in air external to the covering;

c) one or more outlets in the covering, wherein the outlets expel air from inside the covering to outside the covering;

d) one or more pumps in fluid communication with the reservoir, wherein the pumps draw fluid from the aquatic microalgae culture and return the fluid to the culture via a nozzle that sprays the fluid into the airspace above the aquatic microalgae culture.

With respect to embodiments of the enclosed cultivator system, in some embodiments, the reservoir has a growth area of at least about 400 m², for example, at least about 500 m², 600 m², 700 m², 800 m², 900 m², 1000 m², 1200 m², 1500 m², 1800 m², 2000 m², 2500 m² or 3000 m², or more. In some embodiments, the reservoir has a growth area in the range of about 400 m² to about 3000 m².

In some embodiments, the reservoir has a volume capacity of at least about 60,000 L, for example, at least about 75,000 L, 100,000 L, 150,000 L, 200,000 L, 250,000 L, 300,000 L, 350,000 L, 400,000 L, or more. In some embodiments, the reservoir has a volume capacity in the range of about 60,000 L to about 900,000 L, for example, in the range of about 75,000 L to about 400,000 L.

In some embodiments, the reservoir has a depth in the range of about 5 cm to about 40 cm, for example, a depth in the range of about 10 cm to about 35 cm, for example, a depth in the range of about 15 cm to about 30 cm, for example, a depth of about 5 cm, 10 cm, 15 cm, 20 cm, 25 cm, 30 cm, 35 cm, or 40 cm. In some embodiments, the reservoir is configured for a thin-layered smooth sloping cultures (TLSS) with a culture depth of about 5-7 mm). In some embodiments, the reservoir is configured for a thin-layered baffled sloping cultures (TLBS) with a culture depth of about 5-15 mm.

In some embodiments the enclosed cultivator system further comprises a mixer placed in operative communication with the reservoir such that the mixer can mix the aquatic microalgae culture.

In some embodiments, the enclosed cultivator system is a raceway system. For example, in some embodiments, the enclosed cultivator has a width of at least about 16 meters and a length of at least about 100 meters. In some embodiments, the enclosed cultivator has a width of at least about 24 meters and a length of at least about 150 meters. In some embodiments, the enclosed cultivator has an length:width aspect ratio of about 6.25:1.

In some embodiments, the enclosed cultivator is a thin-layered sloping reactor.

In some embodiments, the covering is configured to provide an airspace of about 0.5 meters to about 4.0 meters above the surface of the aquatic microalgae culture, for example, about 1 meter to about 3 meters above the surface of the aquatic microalgae culture, for example, about 0.5, 1.0, 1.5, 2.0, 2.5, 3.0, 3.5 or 4.0 meters above the surface of the aquatic microalgae culture.

In some embodiments, the one or more outlets comprise vents. In some embodiments, the one or more outlets comprise fans.

In some embodiments, the nozzle sprays a cone of liquid at a spray angle from about 30° to about 170°, for example at an angle of about 30°, 60°, 90°, 120°, 150°, 170°, or 180°, as appropriate.

In some embodiments, the one or more pumps is a positive displacement pump, for example, a progressive cavity pump, a peristaltic pump or a lobe pump. In some embodiments, the one or more pumps is an axial flow pump. In some embodiments, the flow rate through the one or more pumps is in the range of about 10 L/min to about 1,000 L/min, for example, about 10 L/min, 20 L/min, 50 L/min, 100 L/min, 200 L/min, 400 L/min, 600 L/min, 800 L/min or 1000 L/min.

In some embodiments, the enclosed cultivator system does not comprise a heat exchanger.

In some embodiments, the enclosed cultivator system further comprises one or more sensors for determining or monitoring one or more parameters within the cultivator, e.g., the ambient temperature of the air above the aquatic culture fluid, the temperature of the aquatic culture fluid, the fluid level or depth of the aquatic culture fluid, the concentration of the microalgae in the culture fluid, the levels or concentrations of nutrients (e.g., salts, CO₂, etc.) in the aquatic culture fluid, the level or concentrations of photosynthetic products (culture density, dissolved O₂, etc.), the speed or flow rate of the one or more fans, the speed or flow rate of the one or more pumps, the speed or flow rates of the one or more mixers, etc.

In some embodiments, the one or more sensors is in operative communication with a computer implemented controller that regulates or modulates the one or more parameters determined or monitored by the one or more sensors, e.g., the ambient temperature of the air above the aquatic culture fluid, the temperature of the aquatic culture fluid, the fluid level or depth of the aquatic culture fluid, the concentration of the microalgae in the culture fluid, the levels or concentrations of nutrients (e.g., salts, CO₂, etc.) in the aquatic culture fluid, the level or concentrations of photosynthetic products (culture density, dissolved O₂, etc.), the speed or flow rate of the one or more fans, the speed or flow rate of the one or more pumps, the speed or flow rates of the one or more mixers, etc.

In some embodiments, the enclosed cultivator system further comprises one or more photovoltaic cells or an array of photovoltaic cells in operative communication with one or more elements in the system that require energy input, e.g., the one or more sensors, the one or more fans, the one or more pumps, the one or more mixers, etc., wherein output current from the one or more photovoltaic cells or array of photovoltaic cells powers the one or more elements in the system that require energy input.

In some embodiments, the enclosed cultivator system is suitable for cultivating Selenestrum, Scenedesmus, Nannochloropsis or Isochrysis, e.g., at a commercial scale and in an energy efficient and economically viable manner.

### DEFINITIONS

The term “microalgae” refers to microphytes, e.g., unicellular eukaryotic species that exist individually or in chains or groups. The microalgae subject to the present concentrating methods generally have an average diameter of about 20 μm or less, for example, about 15 μm, 10 μm, 5 μm, or less. In some embodiments, the microalgae are photosynthetic algae. In some embodiments, the microalgae are of the genus Dunaliella, Chlorella, Tetraselmis, Botryococcus, Haematococcus , Phaeodactylum, Skeletonema, Chaetoceros, Isochrysis, Selenestrum, Scenedesmus, Nannochloropsis, Nannochloris, Pavlova, Nitzschia, Pleurochrysis, Chlamydomas or Synechocystis.

The phrase “cold water species of microalgae” refers to microalgae whose optimal growth temperatures are about 35° C. or less. Exemplary cold water species of algae include without limitation Selenestrum, Scenedesmus, Nannochloropsis or Isochrysis.

The term “large-scale” refers to commercial scale or industrial scale applications of the methods. In some embodiments “large-scale” production of microalgae refers to a culture of at least about 400 L, for example, at least about 500 L, 750 L, or 1000 L, for example, at least about 5000 L, 8000 L, 10000 L, 15000 L, 20000 L, or more.

The term “monoculture” refers to the culture of one species of microorganism (e.g., microalgae) in an aqueous mixture or environment. In some embodiments, a monoculture will have less than 10% contamination, for example, less than 8%, 5%, 3%, 2%, or 1% contamination, with microorganisms not being grown or cultured in the monoculture (i.e., the aqueous mixture contains essentially a monoculture of the microorganism intended to be cultured).

### BRIEF DESCRIPTION OF THE DRAWINGS

FIG. 1 illustrates the dimensions of a typical commercial scale raceway cultivator. The indicated dimensions are in meters. The reservoir, indicated by darker shading, is in a raceway conformation. The lighter shading indicates the perimeter of the covering around the reservoir.

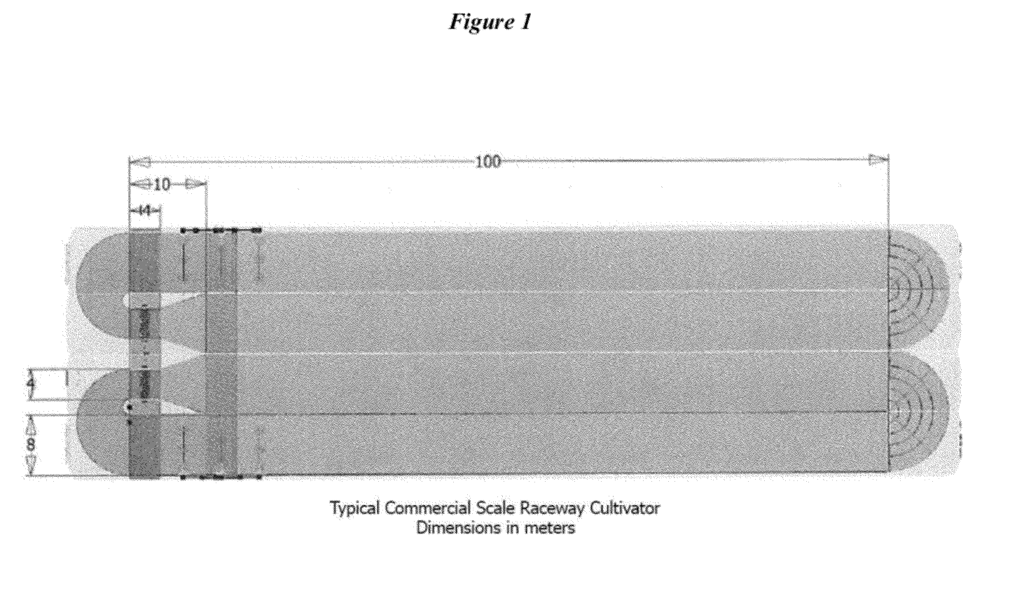

FIG. 2 illustrates an exemplary raceway cultivator with the thermal control mechanisms described herein. First (1) and second (2) inlet fan sets, in this schematic comprising six fans in each set, are positioned at one end of the cultivator. First (7) and second (8) outlet venting sets for discharging moist air to the drier environment outside the cultivator housing, in this schematic comprising six vents in each set, are positioned at the end of the cultivator opposite the inlet fans. First (3) and second (5) sets of positive displacement pumps positioned along the external side of the reservoir, in this schematic a set of six pumps in each set, withdraw aqueous culture from the cultivator reservoir. The withdrawn aqueous culture is returned to the culture in the reservoir through nozzles, e.g., in the form of one or more misted cones. In this schematic the aqueous culture is returned in diffused water spray cones via first (4) and second (6) sets of nine separate nozzles above each reservoir. The pumps and spray nozzles are positioned proximal to the inlet fans. The schematic also depicts the positioning of mixers as darkened rectangles in the internal lanes of the raceways at the end proximal to the inlet fans.

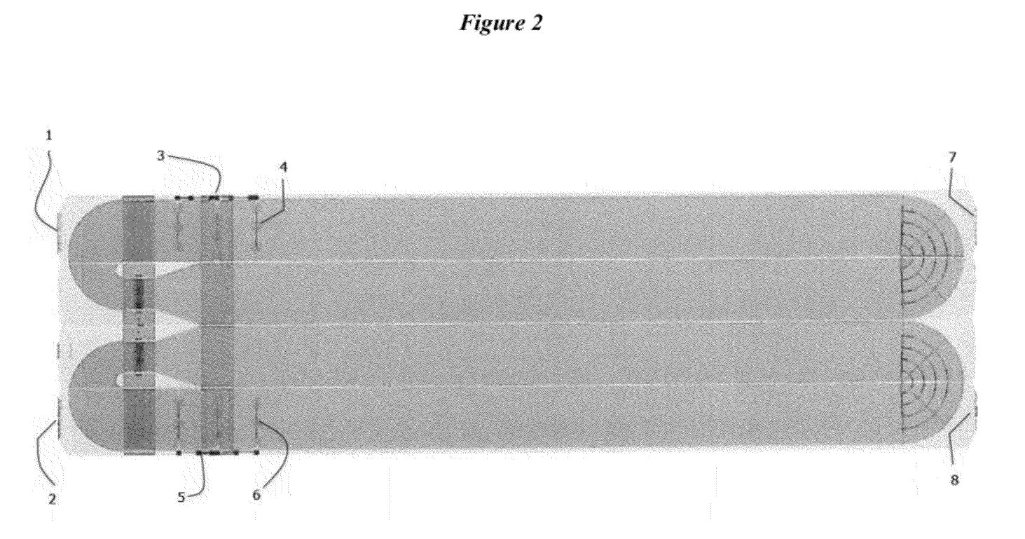

FIG. 3 illustrates an end view of an enclosed raceway cultivator depicting two sets inlet fans positioned in the cultivator covering.

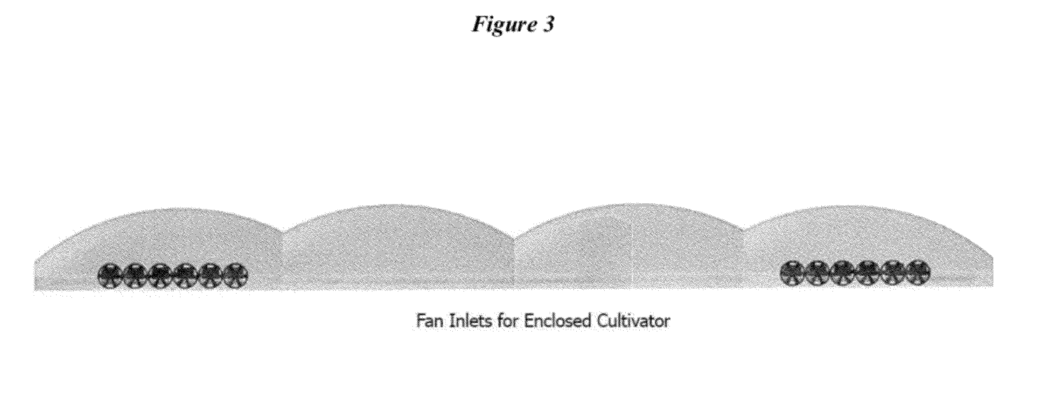

FIG. 4 illustrates an end view of an enclosed raceway cultivator depicting two sets of vent outlets positioned in the cultivator covering. The vents can be louvered or can contain fans that draw air from inside the cultivator and to the outside of the cultivator.

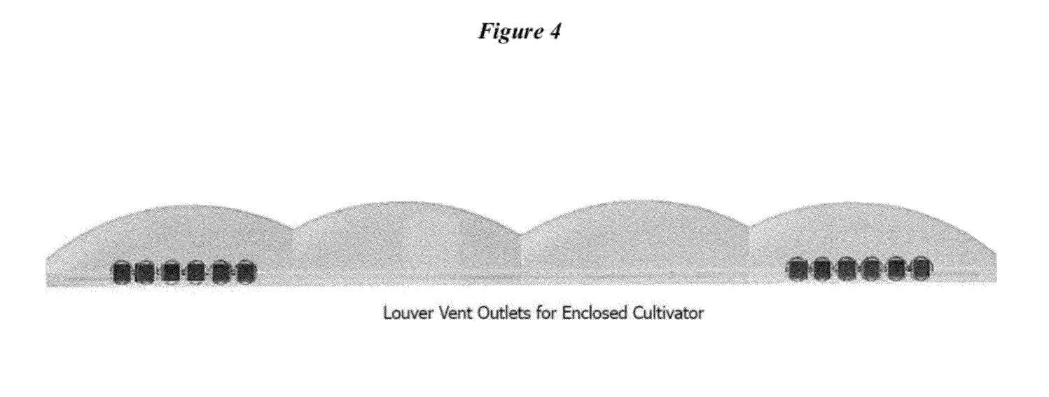

FIG. 5 illustrates a schematic of an interior view of an enclosed cultivator with the temperature regulation mechanisms of the invention. The view depicts spray nozzles and micro algae-laden culture spray cones in the foreground and a set of six inlet fans in the background. The schematic also depicts on the left-hand side, a mixer in the form of rotary paddles.

### DETAILED DESCRIPTION

1. Introduction

The invention relates to the thermal control of an aquatic microalgae culture in an enclosed cultivator. The present invention is based, in part, on the discovery that the temperature of an aquatic microalgae culture in an enclosed cultivator can be actively modulated and/or regulated via evaporative cooling by spraying the liquid of the aquatic microalgae culture in the airspace of the enclosed cultivator above the culture when the airspace is exposed to an airflow from a source of air of relatively lower humidity from outside the enclosed cultivator. In contrast to current methods of temperature regulation, which rely on a heat exchanger and/or conductive heat transfer to regulate or control the temperature of the aquatic microalgae culture, the present methods and systems can be carried out on a commercial scale in an energy efficient and economically viable manner.

Certain species of algae, for example, salt water species including Nannochloropsis and Isochrysis, grow best under cold water conditions. These algae are commercially desirable species due to the high presence of Omega-3 fatty acids in the oil profile. Nannochloropsis sp. are known for EPA. Isochrysis sp. are known for DHA. For example, Table 1 below summarizes the survival temperature extremes and optimal temperature conditions for cold water growing microalgae species of interest.

TABLE 1

| | Temperatures (° C.) | | | | | |
|---|---|---|---|---|---|---|
| | | Min. | | | | Max. |
| | Min. Safe | Safe | Growing | | Esti- | Safe |
| | Survival | Survival | Conditions | | mated | survival |
| Alga Species | (no light) | (light) | Min. | Max. | Optimal | (light) |
| Selenestrum | 15 | 20 | 20 | 35 | 30 | 37 |
| Scenedesmus | 15 | 20 | 20 | 44 | 35 | 45 |
| Nannochloropsis | 8 | 12 | 15 | 25 | 22 | 27 |
| Isochrysis | 8 | 12 | 15 | 30 | 25 | 30 |
| Chlorella | 15 | 20 | 20 | 40 | 35 | 45 |
| Spirulina | | | | | 35-40 | |

The present systems and methods cool an aquatic microalgae culture in an energy efficient manner with a mechanism that is scalable. Direct conductive cooling is energy inefficient and economically non-viable using low cost and cold aquifer water or a separate water loop with a traditional cooling tower. This is because there is a large surface area exposed to the sun and a relatively thin (˜15 cm deep) pool of culture. At times of peak solar insolation, massive amounts of cooling water (on the order of 4000 to 10,000 L/min) are required to keep a full size raceway cultivator (100 m×16 m) sufficiently cool to stay within the optimal growth temperature range for cold water microalgae species. Until the present invention practitioners have avoided the problems associated with growing cold water microalgae species in commercial scale enclosed cultivators and grown species whose temperature range is more suitable for the ambient temperature conditions. For example, Earthrise grows Spirulina in the spring, summer, and early fall in California's Imperial Valley and stops growing in the winter because the ambient temperature is too cold. Similarly, Ami Ben Amotz grows Nannochloropsis in Israel in the winter when the sea water temperatures are cooler and stops growing it in the spring. See, Bio-Fuel and CO₂ Capture by Algae, Agence Nationale Recherche (ANR) Meeting on Third generation Biofuels, Paris, Feb. 5, 2009, on the worldwide web at agence-nationale-recherche.fr/documents/uploaded/2009/6-seminaire-BIOE-Seambiotics_Ami-Ben-Amotz.pdf.

Despite need for energetically and economically viable thermal control of commercial scale enclosed microalgae cultivators, until the present invention, there have been no successful implementations to date in a commercial scale, i.e. one with a growth area of greater than 400 m².

2. Temperature Regulated Enclosed Microalgae Cultivator Systems

The microalgae cultivator is enclosed in that the microalgae cultivation environments is isolated from the environment exterior to the cultivator. The cultivator comprises a reservoir for holding a body of fluid within which microalgae is phototrophically grown. The enclosed cultivator system provides carbon dioxide and nutrients to microalgae. The cultivator can also have a mixer that agitates and/or stirs the microalgae being cultivated in the fluid body. In some embodiments, the mixer is a rotary paddle or an air jet.

The cultivator reservoir is housed under a covering that allows solar radiation to reach the body of fluid. The cultivator covering can be any type of transparent or translucent material including without limitation, plastic and/or glass. The covering or greenhouse enclosing the cultivator reservoir may be a structure where the translucent panels are support by a rigid frame. Exemplary rigid metallic frames are available, e.g., from International Greenhouse Company (exemplary models include without limitation SuperStar Series 3500 Greenhouse, Arch Series 6500 Greenhouse, or Gable Series 7500 Greenhouse). Alternatively, the structure may be an air-supported structure. Air-supported structures that find us are available, e.g., from Yeadon Domes (on the worldwide web at yeadondomes.com). Another energy efficient air-inflated structure that finds use is available from Airstream Innovations (on the worldwide web at airstreaminovations.com/products.html). The covering from said structures could be, e.g., reinforced plastic. Reinforced plastic for cultivator coverings that find use are available, e.g., from PicPlast Ltd (exemplary models include SolarRoof 172 or Solarig 140N) (on the worldwide web at pic-plast.com). The covering generally has approximately the same outer dimensions as the cultivator reservoir. The greenhouse or cultivator can be a rigid structure, e.g., the covering of the cultivator can be supported by a rigid framework, or an inflated structure, e.g., where the covering is maintained by a pressurization by a fan.

The enclosed microalgae cultivators of the present invention are suitable for commercial scale cultivation of microalgae. For example, in some embodiments, the reservoir has a growth area of at least about 400 m², for example, at least about 500 m², 600 m², 700 m², 800 m², 900 m², 1000 m², 1200 m², 1500 m², 1800 m², 2000 m², 2500 m² or 3000 m², or more. In some embodiments, the reservoir has a growth area in the range of about 400 m² to about 3000 m².

In some embodiments, the reservoir has a volume capacity of at least about 60,000 L, for example, at least about 75,000 L, 100,000 L, 150,000 L, 200,000 L, 250,000 L, 300,000 L, 350,000 L, 400,000 L, or more. In some embodiments, the reservoir has a volume capacity in the range of about 60,000 L to about 900,000 L, for example, in the range of about 75,000 L to about 400,000 L.

In some embodiments, the aquatic microalgae culture has a depth in the range of about 5 cm to about 40 cm, for example, a depth in the range of about 10 cm to about 35 cm, for example, a depth in the range of about 15 cm to about 30 cm, for example, a depth of about 5 cm, 10 cm, 15 cm, 20 cm, 25 cm, 30 cm, 35 cm, or 40 cm. In some embodiments, the reservoir is configured for a thin-layered smooth sloping cultures (TLSS) with a culture depth of about 5-7 mm). In some embodiments, the reservoir is configured for a thin-layered baffled sloping cultures (TLBS) with a culture depth of about 5-15 mm. See, e.g., Grobbelaar, et al., “Variation in some photosynthetic characteristics of microalgae cultured in outdoor thin-layered sloping reactors,” J App Phycol (1995) 7:175-184 and Setlik, et al., Alol Stud (Trebon) (1970) 1:111.

In some embodiments, the enclosed cultivator system further comprises a mixer placed in operative communication with the reservoir such that the mixer can mix the aquatic microalgae culture.

The enclosed cultivator system can be any appropriate shape, including without limitation rectangular, square, oval or circular.

In some embodiments, the enclosed cultivator system is a raceway system. For example, in some embodiments, the enclosed cultivator has a width of at least about 16 meters and a length of at least about 100 meters. In some embodiments, the enclosed cultivator has a width of at least about 24 meters and a length of at least about 150 meters. In some embodiments, the enclosed cultivator has an length:width aspect ratio of about 6.25:1.

In some embodiments, the enclosed cultivator system is a thin-layered sloping reactor. See, e.g., Grobbelaar, et al., “Variation in some photosynthetic characteristics of microalgae cultured in outdoor thin-layered sloping reactors,” J App Phycol (1995) 7:175-184 and Setlik, et al., Alol Stud (Trebon) (1970) 1:111.

Generally, the enclosed air volume atop the aquatic culture is minimized to reduce or minimize the amount of required air turnover. In some embodiments, the covering is configured to provide an airspace of about 0.5 meters to about 4.0 meters above the surface of the aquatic microalgae culture, for example, about 1 to 3 meters or about 1 to 1.5 meters above the surface of the aquatic microalgae culture, for example, about 0.5, 1.0, 1.5, 2.0, 2.5, 3.0, 3.5 or 4.0 meters above the surface of the aquatic microalgae culture.

Airflow in the airspace above the fluid or aquatic microalgae culture is generated using any method known in the art. For example, one or more fans with access to air outside the cultivator can be placed in the covering. The intake inlets comprising fans draw air in from outside the cultivator housing into the airspace enclosed by the housing. This brings in air of lower relative humidity (RH) from outside the cultivator into the cultivator.

The intake inlets comprising fans can be placed on one end of the cultivator or on one or more sides of the cultivator. The intake fans can be placed low, i.e., close to the level of the reservoir or body of fluid, or place in a higher positions, e.g., 0.5, 1.0, 1.5, 2.0, 2.5 meters above the reservoir or body of fluid. In some embodiments, multiple fans can be placed on one side of the cultivator and at different heights above the reservoir or body of fluid. The fans are in operable communication with a motor, for example a uniform speed motor or a variable speed motor, as appropriate. The volume of air down across the length or width of the cultivator is modulated or adjusted, changing the air velocity over the cultivator as appropriate. This assures that the air is less than 100% RH so that evaporation can continue.

The number of inlets and fans in the cultivator will depend on the amount of air flow needed and the size and shape of the cultivator. In some embodiments, a cultivator can have 1, 2, 3, 4, 5, 6, 7, 8, 9, 10, 11, 12, or more inlet fans, as needed or desired. The inlets can be evenly spaced across a side of the cultivator, or arranged in sets, for example, concentrated at the end of a length of the reservoir. The size of the inlets can also vary depending on the amount of air flow needed. In some embodiments, each inlet has an average diameter of about 0.2, 0.3, 0.4, 0.5, 0.6, 0.7, 0.8, 0.9, 1.0, 1.2 meters, or more, as needed or desired.

In the case of a rigid greenhouse, the one or more fans move air throughout the airspace above the aquatic culture, e.g., for ventilation. In the case of an air-inflated greenhouse, the one or more fans maintain the internal pressure to support the flexible covering. Even with an air-inflated greenhouse, the one or more fans can be driven at such a speed that the air delivery is sufficient to move fresh air through the greenhouse or cultivator. In some embodiments, the airflow across the airspace above the aquatic microalgae culture is in the range of from about 0.1 m/s to about 25 m/s, for example, about 0.5 m/s to about 8 m/s, or about 1 m/s to about 4 m/s.

The one or more inlets can also have a screen or a filter to reduce, minimize or prevent contamination of the culture in the cultivator. The screen or filter can be attached to the surface of the one or more inlets inside the cultivator.

Air of relative higher humidity is exhausted or expelled through one or more outlets in the cultivator covering. The outlets can comprise exhaust vents or fans that draw air from the inside of the cultivator and exhaust the air to the outside of the cultivator. This releases or forces out air of higher relative humidity inside the cultivator to the outside of the cultivator. The venting or exhausting outlets, with or without fans, can be placed on one end of the cultivator or on one or more sides of the cultivator. The venting or exhausting outlets, with or without fans, can be placed low, i.e., close to the level of the reservoir or body of fluid, or place in a higher positions, e.g., 0.5, 1.0, 1.5, 2.0, 2.5 meters above the reservoir or body of fluid. In some embodiments, multiple venting or exhausting outlets, with or without fans, can be placed on the side of the cultivator and at different heights above the reservoir or body of fluid. In embodiments that have outlet fans, the outlet exhaust fans are in operable communication with a motor, for example, a uniform speed motor or a variable speed motor, as appropriate. In some embodiments, the inlets and outlets in the cultivator covering for passive or active airflow are positioned on opposite sides of the cultivator. The opposite sides can be across the width or length of the cultivator. In one embodiment, the inlets and outlets are positioned at opposite sides across the length of the cultivator.

The number of outlets (containing vents or fans) in the cultivator will depend on the amount of air flow needed and the size and shape of the cultivator. In some embodiments, a cultivator can have 1, 2, 3, 4, 5, 6, 7, 8, 9, 10, 11, 12, or more outlets, as needed or desired. The outlets can be evenly spaced across a side of the cultivator, or arranged in sets, for example, concentrated at the end of a length of the reservoir. The size of the outlets can also vary depending on the amount of air flow needed. In some embodiments, each outlet has an average diameter of about 0.2, 0.3, 0.4, 0.5, 0.6, 0.7, 0.8, 0.9, 1.0, 1.2 meters, or more, as needed or desired. The number and/or size of outlets can, but need not be, matched to the number and/or size of inlets.

The one or more outlets can also have a screen or a filter to reduce, minimize or prevent contamination of the culture in the cultivator. The screen or filter can be attached to the surface of the one or more outlets inside the cultivator.

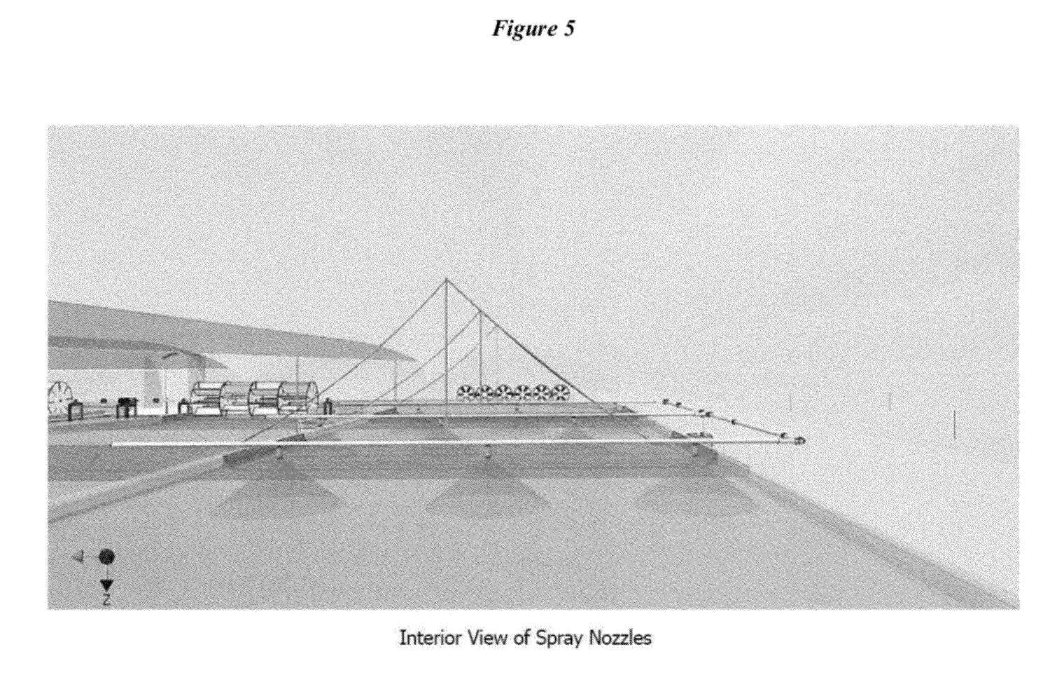

The cultivator systems of the invention comprise one or more pumps in fluid communication with aquatic microalgae culture in the reservoir. The pumps draw microalgae-laden culture fluid from the reservoir and spray the fluid through a nozzle into the airspace above the culture fluid, which effects evaporation of the culture fluid and cooling of the airspace and liquid culture. The one or more pumps can be positioned near a inlet or intake fan, which facilitates blowing of the sprayed culture into the enclosed cultivator. The pumps can also be directly connected to the spray nozzle or in close proximity to the spray nozzle to minimize or eliminate tubing between the pump and spray nozzle. In some embodiments, a framework of pipes or tubing in fluid communication with the one or more pumps and the one or more nozzles is arranged above the surface of the culture fluid. See, e.g., FIG. 5. The framework of pipes or tubing can be suspended from the framework of the covering or supported from below, as desired.

In some embodiments, the one or more pumps is a positive displacement pump, for example, a progressive cavity pump or a lobe pump. In some embodiments, the one or more pumps is an axial flow pump. Exemplary pumps that find use include without limitation a positive displacement pump including a lobe pump (e.g., available from Waukesha Cherry-Burrell), peristaltic pump (e.g., available from Watson-Marlow, Larox, or Blackmer), or progressive cavity (PC) pump (e.g., available from Seepex, Netzsch, or Moyno). In other embodiments, an axial flow pump is used. Axial flow pumps provide high flow velocity while having low differential pressure capability. Exemplary pumps are available from Van Ness Lo-Lift, Vertiflo, Gator Pump, and Gould.

Generally, the pumps and spray nozzles are suitable to withstand a flow rate in the range of about 10 L/min to about 1,000 L/min, for example, about 10 L/min, 20 L/min, 50 L/min, 100 L/min, 200 L/min, 400 L/min, 600 L/min, 800 L/min or 1000 L/min. The flow rate will depend, in part, on the type and quantity of spray nozzle used, the number of pumps in operation, and the flow rate of fluid through the pumps.

Nozzles that find use spray the microalgae culture into the airspace within the enclosed cultivator as a fine mist, oftentimes in the shape of a cone. The nozzle can aim the spray in any direction sufficient to allow the microalgae-laden spray to return to the culture fluid. For example, the nozzle can be aimed up away from the culture fluid (e.g., 180° from the culture fluid), across the culture fluid (e.g., 90° over the culture fluid), or directly into the culture fluid (e.g., 0° into the culture fluid). The nozzle can be positioned at a height above the fluid sufficient spray a cone of liquid above the culture. For example, the nozzle can be positioned at a height of at least about 0.3, 0.5, 0.8, 1.0, 1.3, 1.5, 1.8, 2.0 meters, or more, above the liquid culture. In some embodiments, the nozzle sprays a cone of liquid at a spray angle from about 30° to about 170°, for example at an angle of about 30°, 60°, 90°, 120°, 150°, 170°, or 180°, as appropriate. Exemplary nozzles include those commercially available from, e.g., BETE Fog Nozzle, Inc., Greenfield, Mass. (on the worldwide web at BETE.com).

The enclosed microalgae cultivators of the present invention further comprise one or more sensors for determining or monitoring one or more parameters within the cultivator, e.g., the ambient temperature of the air above the aquatic culture fluid, the relative humidity of the air above the aquatic culture fluid, the temperature of the aquatic culture fluid, the fluid level or depth of the aquatic culture fluid, the concentration of the microalgae in the culture fluid, the levels or concentrations of nutrients (e.g., salts, CO₂, etc.) in the aquatic culture fluid, the level or concentrations of photosynthetic products (culture density, dissolved O₂, etc.), the speed or flow rate of the one or more fans, the speed or flow rate of the one or more pumps, the speed or flow rates of the one or more mixers, etc.

In some embodiments, a temperature sensor is mounted in the area where the ambient temperature is to be determined, e.g., outside or on the outside surface of the covering, in the airspace in the inside of the cultivator or on the inside surface of the covering, or within the culture fluid. Other sensors can be placed in the flow paths of the one or more pumps, one or more intake fans or one or more mixers.

The concentration of the culture, depth of the culture, and nutrient concentrations of the culture can be monitored by any method known in the art. For example, the concentration of the culture can be determined manually, with an optimal density meter, or with a coriolis mass flow meter (e.g., available from GE Rheonik, Micromotion, ABB, Brooks, Krohne, Yokogawa, or Endress+Hauser). A level regulation device such as a float switch can be employed to monitor the culture depth and provide makeup water to counteract the losses due to evaporation. Nutrient concentration within the culture can be monitored via either on-line or off-line analysis, e.g., using methods documented by the American Water Works Association (“AWWA”).

The one or more sensors can be in operative communication with a computer implemented controller that regulates or modulates the one or more parameters determined or monitored by the one or more sensors. For example, should the temperature in the enclosed cultivator rise above a threshold temperature, the temperature sensors in the cultivator communicate this information to the computer, which in turn communicates with regulators of the intake fans and/or the pumps to increase air and/or water flow, respectively, thereby effecting increased evaporative cooling of the culture. Should the temperature in the enclosed cultivator fall below a threshold temperature, the temperature sensors in the cultivator communicate this information to the computer, which in turn communicates with regulators of the intake fans and/or the pumps to decrease or stop air and/or water flow, respectively, thereby effecting decreased evaporative cooling of the culture.

In some embodiments, the enclosed cultivator system further comprises one or more photovoltaic cells or an array of photovoltaic cells in operative communication with one or more elements in the system that require energy input, e.g., the one or more sensors, the one or more fans, the one or more pumps, the one or more mixers, etc., wherein output current from the one or more photovoltaic cells or array of photovoltaic cells powers the one or more elements in the system that require energy input. The photovoltaic cells or array of photovoltaic cells can be placed on or near the enclosed cultivator. Preferably the photovoltaic cells are placed so that they do not interfere with solar radiation reaching the aquatic microalgae culture in the reservoir.

3. Methods of Regulating Temperature of an Aquatic Microalgae Culture in an Enclosed Cultivator

The invention further provides methods of using the enclosed microalgae cultivators described herein. Generally, the present processes leverage evaporative cooling to regulate the temperature within an enclosed cultivator, and do not involve a heat exchanger or conductive heat transfer.

An airflow is maintained across the airspace above the aquatic microalgae culture. The airflow can be passively or actively maintained, as needed. Active airflow can be maintained using any method known in the art. In one embodiment, air intake inlets in the cultivator covering comprising fans that draw in air from outside the cultivator into the cultivator are used. For example, should temperatures within the cultivator rise above a threshold temperature, the speed of the one or more fans in the air intake inlets can increase so that airflow increases, thereby effecting evaporative cooling of the temperature inside the cultivator, e.g., in the airspace and/or in the liquid culture. The one or more fans operating in the intake inlets bring fresh air from the exterior environment into the cultivator and discharge through an outlet. In some embodiments, the inlets and outlets in the cultivator covering for passive or active airflow are positioned on opposite sides of the cultivator. As discussed above, the outlets can comprise exhaust vents or fans that draw air from the inside of the cultivator to the outside of the cultivator. The fan speed and/or number of operating fans can be modulated (increased or decreased) to increase the flow of external air into the cultivator. When the external air drawn in has a lower relative humidity (RH; low moisture content), water evaporates from the microalgae culture to bring this air into equilibrium with the culture. The act of evaporation results in cooling of the culture total. The rate of evaporation of the culture fluid can be increased by increasing the airflow across the airspace in the enclosed cultivator. The lower RH provides a driving force for evaporation as the water will evaporate until the moisture in the gas (air) above the cultivator is equal at a 100% RH (maximum amount of water the air can hold at a given temperature). The present methods generally supply an excess quantity of air, because air at 100% RH does not allow for evaporation. Therefore, the rate of airflow in the airspace above the aquatic microalgae culture is sufficient to maintain the air in the airspace below 100% RH, for example, at about 97%, 95%, 90%, 85%, 80%, 75% or 70% RH. In some embodiments, the airflow across the airspace above the aquatic microalgae culture is in the range of from about 0.1 m/s to about 25 m/s, for example, about 0.5 m/s to about 8 m/s, or about 1 m/s to about 4 m/s.

The rate of evaporation of the culture fluid can be further increased by increasing the surface area of the culture fluid. This can be accomplished using any known methods in the art. In one embodiment, the invention utilize a secondary culture circulation loop, where the culture is microalgae-laden water. For example, microalgae culture fluid can be pumped through a nozzle and sprayed into the airspace above the microalgae culture fluid, as described herein.

Microalgae culture fluid is drawn from the culture in the reservoir, put through one or more pumps to boost its pressure, and sprayed back into the culture fluid through a nozzle. The spray also further increases the air flow above the culture fluid. Since lobe, peristaltic and progressive cavity pumps are positive displacement, changing the pump's motor speed directly changes the volumetric flow rate. This can be used to modulate the water drop area exposed to the moving air and results in reduction in energy to cool the system.

Fluid from the nozzle is sprayed back into the cultivator with dryer exterior air flowing through the spray pattern. The air flow and the spray enhances evaporation and, in turn, results in cooling of the main volume within the culture. Increasing or decreasing the pump speed, or increasing or decreasing the number of operating pumps, modulates the amount of exposed water. This varies the heat transfer rate and, thus, enables the rate of cooling to be controlled.

In some embodiments, the flow rate of the microalgae culture through the one or more pumps and spray nozzles is in the range of about 10 L/min to about 1,000 L/min, for example, about 10 L/min, 20 L/min, 50 L/min, 100 L/min, 200 L/min, 400 L/min, 600 L/min, 800 L/min or 1000 L/min. The flow rate will depend, in part, on the type and quantity of spray nozzle used, the number of pumps in operation, and the flow rate of fluid through the pumps. In some embodiments, the nozzle sprays a cone of liquid at a spray angle from about 30° to about 170°, for example at an angle of about 30°, 60°, 90°, 120°, 150°, 170°, or 180°, as appropriate.

In some embodiments, the methods further comprise sensing the ambient temperature, e.g., of the airspace inside of the cultivator, of the microalgae culture fluid, of the air outside of the cultivator. In some embodiments, the methods further comprise sensing one or more parameters selected from the ambient temperature of the air above the aquatic culture fluid, the relative humidity of the air above the aquatic culture fluid, the temperature of the aquatic culture fluid, the fluid level or depth of the aquatic culture fluid, the concentration of the microalgae in the culture fluid, the levels or concentrations of nutrients (e.g., salts, CO₂, etc.) in the aquatic culture fluid, the level or concentrations of photosynthetic products (culture density, dissolved O₂, etc.), the speed or flow rate of the one or more fans, the speed or flow rate of the one or more pumps, the speed or flow rates of the one or more mixers. The sensing can be done using any method in the art. The sensing of the one or more parameters can be performed prior to operation of the fans and/or pumps, concurrent with the operation of the fans and/or pumps, and/or prior to increasing or decreasing the flow rates through the fans and/or pumps. In some embodiments, sensing can be performed during predetermined intervals throughout a 24-hour period, e.g., every 15 minutes, every 30 minutes, every hour, every 2 hours, every 3 hours, every 4 hours, every 6 hours, every 12 hours, or more or less often, as needed or desired. For example, sensing of the one or more parameters may be performed more often during the daylight hours, and particularly in the afternoon, when the effect of solar radiation are the most intense. Sensors can be positioned as described above, in the airspace or liquid culture within the cultivator and/or just outside the cultivator.

Regulation or modulation of temperature within the enclosed cultivator can be achieved by regulating the airflow within the cultivator above the liquid culture and the fluid flow through the pump, and therefore the quantity of spraying. Should the temperatures rise above a predetermined threshold temperature, the speed of the intake fans and/or the pumps forcing the culture fluid is increased to increase evaporative cooling. Should the temperatures fall below a predetermined threshold temperature or the level of the culture fluid fall below a predetermined threshold depth, the speed of the intake fans and/or the pumps forcing the culture fluid can be decreased or stopped to decrease or stop evaporative cooling, as needed or desired. The flow rates of the fans and the pumps can be coordinated to concurrently increase and/or decrease or the flow rates of the fans and the pumps can be independent of one another.

Using the evaporative cooling strategies of the present invention, the temperature within the aquatic microalgae culture can be cooled at least about 1° C., 2° C., 3° C., 4° C., 5° C., 6° C., 7° C., 8° C., 9° C., 10° C., 11° C., 12° C., 13° C., 14° C., 15° C., or more, as needed or desired. The environment within the cultivator can be maintained at a temperature that is in equilibrium with the ambient temperature external to the enclosed cultivator, or that is at least about 1° C., 2° C., 3° C., 4° C., 5° C., 6° C., 7° C., 8° C., 9° C., 10° C., 11° C., 12° C., 13° C., 14° C., 15° C., or more, cooler than the ambient temperature external to the enclosed cultivator. The minimum temperature that can be attained corresponds to the current wet-bulb temperature which is, in turn, a function of the temperature and the relative humidity.

The processes can successfully maintain an aquatic microalgae culture at temperatures suitable for culture of cold water microalgae species, e.g., including without limitation Selenestrum, Scenedesmus, Nannochloropsis or Isochrysis. Generally, the temperature of the aquatic microalgae culture is maintained at temperatures that are at or below about 35° C. In some embodiments, the aquatic microalgae culture is maintained at a temperature in the range of about 15° C. to about 35° C., for example, from 20-35° C., 15-25° C. or 15-30° C. In some embodiments, the aquatic microalgae culture is maintained at a temperature of about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., or higher or lower, as need or desired, depending on the species of microalgae being cultivated.

In some embodiments, the one or more sensors, the one or more fans, the one or more pumps, the one or more mixers, etc., are powered by output current from one or more photovoltaic cells or array of photovoltaic cells in operative communication with the sensors, fans, pumps, and/or mixers.

Using sensors, computers, regulators and photovoltaic cells in operative communication with each other, the evaporative cooling processes of the present invention are well suited to operate in an automated and energy efficient manner. For example, the enclosed cultivators described herein can contain one or more sensors in operative communication with a computer implemented controller that regulates or modulates the one or more parameters determined or monitored by the one or more sensors, e.g., the ambient temperature of the air above the aquatic culture fluid, the temperature of the aquatic culture fluid, the fluid level or depth of the aquatic culture fluid, the concentration of the microalgae in the culture fluid, the levels or concentrations of nutrients (e.g., salts, CO₂, etc.) in the aquatic culture fluid, the level or concentrations of photosynthetic products (culture density, dissolved O₂, etc.), the speed or flow rate of the one or more fans, the speed or flow rate of the one or more pumps, the speed or flow rates of the one or more mixers, etc.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of regulating the temperature of an aquatic microalgae culture in an enclosed cultivator comprising:
a) maintaining airflow in the airspace within the enclosed cultivator above the aquatic microalgae culture by taking in air from a source external to the enclosed cultivator and exhausting air from the airspace above the aquatic culture to the outside the enclosed cultivator, thereby effecting evaporative cooling; and
b) spraying the aquatic microalgae culture in the airspace above the aquatic microalgae culture; thereby increasing the surface area of water exposed to the airflow and enhancing the effect of evaporative cooling, whereby the temperature of the aquatic microalgae culture in the enclosed cultivator is regulated.

2. The method of claim 1, wherein the aquatic microalgae culture is maintained at a temperature at or below about 35° C.

3. The method of claim 1, wherein the aquatic microalgae culture is maintained at a temperature in the range of about 15° C. to about 35° C.

4. The method of claim 1, wherein the aquatic microalgae culture has a growth area of at least about 400 m².

5. The method of claim 1, wherein the enclosed cultivator is a raceway system.

6. The method of claim 5, wherein the enclosed cultivator has a width of at least about 16 meters and a length of at least about 100 meters.

7. (canceled)

8. The method of claim 1, wherein the aquatic microalgae culture is sprayed through a nozzle that expels a cone of liquid at a spray angle from about 30° to about 170°.

9. The method of claim 1, wherein regulating the temperature of the aquatic microalgae culture in the enclosed cultivator does not involve conductive heat transfer.

10. The method of claim 1, further comprising the step of determining the ambient temperature and/or the relative humidity within the enclosed cultivator before spraying the aquatic microalgae culture.

11. The method of claim 1, wherein the flow of the air in the airspace above the aquatic microalgae culture and the flow of the spraying are powered by the output current of a photovoltaic cell.

12. A method of enhancing evaporative cooling of an aquatic microalgae culture in an enclosed cultivator comprising spraying the aquatic microalgae culture in the airspace above the aquatic microalgae culture; thereby enhancing the effect of evaporative cooling.

13. An enclosed cultivator system, comprising:
a) a covering over a reservoir, wherein the reservoir is configured for maintaining an aquatic microalgae culture;
b) one or more inlets in the covering, wherein the inlets comprise a fan for taking in air external to the covering;
c) one or more outlets in the covering, wherein the outlets expel air from inside the covering to outside the covering;
d) one or more pumps in fluid communication with the reservoir, wherein the pumps draw fluid from the aquatic microalgae culture and return the fluid to the culture via a nozzle that sprays the fluid into the airspace above the aquatic microalgae culture.

14. The enclosed cultivator system of claim 13, wherein the enclosed cultivator has a growth area of at least about 400 m².

15. The enclosed cultivator system of claim 13, wherein the enclosed cultivator is a raceway system.

16. The enclosed cultivator system of claim 13, wherein the enclosed cultivator has a width of at least about 16 meters and a length of at least about 100 meters.

17. (canceled)

18. The enclosed cultivator system of claim 13, wherein the covering is configured to provide an airspace of about 1 meter to about 3 meters above the surface of the aquatic microalgae culture.

19. The enclosed cultivator system of claim 13, wherein the reservoir has a depth of about 15-30 cm.

20. The enclosed cultivator system of claim 13, wherein the one or more outlets comprise vents.

21. The enclosed cultivator system of claim 13, wherein the one or more outlets comprise fans.

22. The enclosed cultivator system of claim 13, wherein the nozzle sprays a cone of liquid at a spray angle from about 30° to about 170°.

23-30. (canceled)
